SEQUENCE LISTING (10 sequences)

SEQ ID NO: 1 - DNA, 17 bp, Artificial
  a forward primer for amplifying a target sequence of ABO blood type gene of human being
taggaaggatgtccgcg

SEQ ID NO: 2 - DNA, 25 bp, Artificial
  a reverse primer for amplifying a target sequence of ABO blood type gene of human being
ttcttgatggcaaacacagttaacc

SEQ ID NO: 3 - DNA, 21 bp, Artificial
  an origo DNA for controlling non-specific amplification other than a target sequence
gtgcggccacatggagctggc

SEQ ID NO: 4 - DNA, 17 bp, Artificial
  a forward primer for amplifying a target sequence of ALDH2 gene of human being
ggctgcaggcatacact

SEQ ID NO: 5 - DNA, 17 bp, Artificial
  a reverse primer for amplifying a target sequence of ALDH2 gene of human being
ggcaggtcctgaacctc

SEQ ID NO: 6 - DNA, 21 bp, Artificial
  an origo DNA for controlling non-specific amplification other than a target sequence
agcagttgaccctgtaatttg

SEQ ID NO: 7 - DNA, 24 bp, Artificial
  a forward primer for amplifying a target sequence of dystrophin gene of human being
aggcttgaaagggcaagtagaagt

SEQ ID NO: 8 - DNA, 21 bp, Artificial
  a reverse primer for amplifying a target sequence of dystrophin gene of human being
gctgatctgctggcatcttgc

SEQ ID NO: 9 - DNA, 26 bp, Artificial
  an origo DNA for controlling non-specific amplification other than a target sequence
gactttttctcaacacttttgccatc

SEQ ID NO: 10 - DNA, 25 bp, Artificial
  an origo DNA for controlling non-specific amplification other than a target sequence
tggaagaaaatgggatgtggtagaa

(19) United States
(12) Patent Application Publication
(10) Pub. No.: US 2012/0322111 A1
(43) Pub. Date: Dec. 20, 2012

(54) METHOD FOR AMPLIFYING A TARGET SEQUENCE INCLUDED IN A DOUBLE-STRANDED DNA

(75) Inventors: Miho Hayashi, Ehime (JP); Hidenobu Yaku, Osaka (JP)

(73) Assignee: PANASONIC CORPORATION, Osaka (JP)

(21) Appl. No.: 13/591,918

(22) Filed: Aug. 22, 2012

Related U.S. Application Data
(63) Continuation of application No. PCT/JP10/01297, filed on Feb. 25, 2010.

(51) Int. Cl. C12P 19/34

(52) U.S. Cl. 435/91.2

(57) ABSTRACT

the present invention provides an amplification method capable of inhibiting the generating of the undesired amplified double-stranded DNA sequence. In the present method, DNA polymerase, deoxynucleoside triphosphate, the double-stranded DNA, a forward primer, a reverse primer, and a first block nucleic acid are mixed so as to amplify the double-stranded target sequence with use of a polymerase chain reaction. The first block nucleic acid does not serve as an origin for the elongation reaction with the DNA polymerase. The first block nucleic acid is complementary with a part of the third non-amplified sequence which is interposed between the 5′ end and the complimentary single-stranded target sequence. Due to the first block nucleic acid, the generating of the undesired amplified double-stranded DNA sequence is inhibited.

### CROSS REFERENCE TO RELATED APPLICATIONS

This application is a continuation of International Application No. PCT/JP2010/001297, filed on Feb. 25, 2010, the disclosure of the application is incorporated by reference herein.

### TECHNICAL FIELD

The present invention relates to a method for amplifying a target sequence included in a double-stranded DNA.

### BACKGROUND

A polymerase chain reaction method (hereinafter, referred to as “PCR method”) is a representative method for amplifying a target sequence 1 included in a double-stranded DNA consisting of a first single-stranded DNA 6 and a second single-stranded DNA 7.

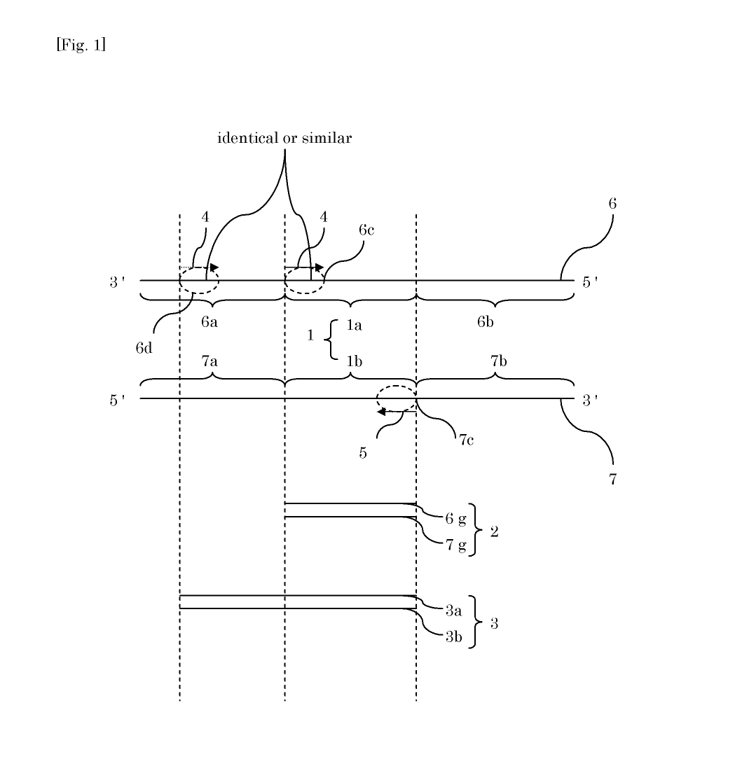

The PCR method is briefly described below with reference to FIG. 1.

The first single-stranded DNA 6 consists of the 3′ end—a first sequence 6a—a single-stranded target sequence 1a—a second sequence 6b—5′ end. The second single-stranded DNA 7 consists of the 5′ end-a third sequence 7a—a complementary single-stranded target sequence 1b—a fourth sequence 7b—3′ end. The complementary single-stranded target sequence 1b, the third sequence 7a, and the fourth sequence 7b are complementary to the single-stranded target sequence 1a, the first sequence 6a, and the second sequence 6b, respectively.

First, DNA polymerase, deoxynucleoside triphosphate, the double-stranded DNA 1, a forward primer 4, and a reverse primer 5 are mixed to prepare a mixture.

The forward primer 4 consists of a nucleic acid having 5-20 bases. The forward primer 4 is complementary to a sequence 6c located at the 3′ end side of the single-stranded target sequence 1a. The reverse primer 5 consists of a nucleic acid having 5-20 bases. The reverse primer 5 is complementary to a sequence 7c located at 3′-end side of the complementary single-stranded target sequence 1b. Accordingly, the forward primer 4 and the reverse primer 5 bind to the sequence 6c and the sequence 7c, respectively.

Then, the mixture is heated at a temperature of 94 degrees Celsius-100 degrees Celsius for 1-100 seconds. Subsequently, the mixture was cooled at a temperature of 50-70 degrees Celsius for 1-100 seconds. The heating and the cooling are repeated to obtain an amplified double-stranded DNA sequence 2. The amplified double-stranded DNA sequence 2 consists of an amplified single-stranded target sequences 6g identical to the single-stranded target sequence 1a and an amplified complementary single-stranded target sequences 7g identical to the complementary single-stranded target sequence 1b. The first sequence 6a, the second sequence 6b, the third sequence 7a, and the fourth sequence 7b are not amplified.

Patent Literature 1, Patent Literature 2, and Patent Literature 3 may be relevant to the present invention.

### CITATION LIST

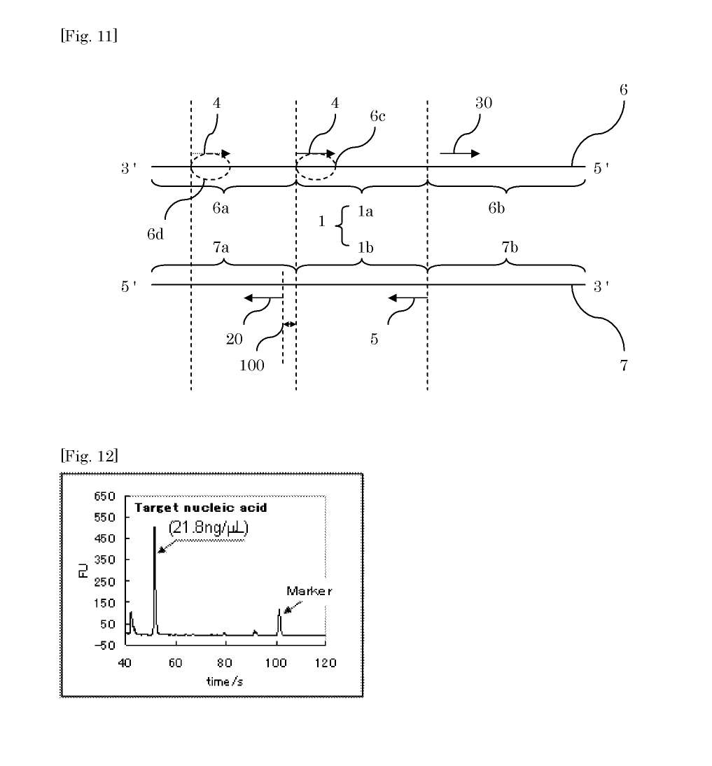

### Patent Literature

[Patent Literature 1] Japanese Patent Laid-Open Publication No. H 5-199900

[Patent Literature 2] Japanese Unexamined Patent Application Publication (Translation of PCT Application) No. 2003-534772

[Patent Literature 3] Japanese Unexamined Patent Application Publication (Translation of PCT Application) No. H 8-508636 (Particularly, FIG. 3 and FIG. 12)

### SUMMARY OF INVENTION

### Technical Problem

As shown in FIG. 1, when the first sequence 6a contains a sequence 6d identical to the sequence 6c, the forward primer 4 binds not only to the sequence 6c but also to the sequence 6d. When the first sequence 6a contains a sequence 6d similar to the sequence 6c, the forward primer 4 may bind not only to the sequence 6c but also to the sequence 6d by mistake.

Accordingly, the amplified double-stranded DNA sequence thus obtained includes not only the desirable amplified double-stranded DNA sequence 2 but also an undesirable amplified double-stranded DNA sequence 3. The undesirable amplified double-stranded DNA sequence 3 is the resultant products of a non-specific amplification. The undesirable amplified double-stranded DNA sequence 3 consists of an undesirable amplified single-stranded DNA 3a and an undesirable amplified complementary single-stranded DNA 3b.

The purpose of the present invention is to provide an amplification method capable of suppressing the generation of the undesirable amplified double-stranded DNA sequence 3.

### Solution to Problem

The present invention solving the problem is a method for amplifying a double-stranded target sequence included in a double-stranded DNA, the method comprising steps of:

a step (a) of mixing DNA polymerase, deoxynucleoside triphosphate, the double-stranded DNA, a forward primer, a reverse primer, and a first block nucleic acid 20, and amplifying the double-stranded target sequence using a PCR method, wherein,

the double-stranded DNA consists of a first single-stranded DNA 6 and a second single-stranded DNA 7,

the double-stranded target sequence consist of a single-stranded target sequence 1a and a complementary single-stranded target sequence 1b,

the first single-stranded DNA 6 consists of the 3′ end—a first sequence 6a—the single-stranded target sequence 1a—a second sequence 6b—5′ end,

the second single-stranded DNA 7 consists of the 5′-end—a third sequence 7a—the complementary single-stranded target sequence 1b—a fourth sequence 7b—3′-end,

the complementary single-stranded target sequence 1b, the third sequence 7a, and the fourth sequence 7b are complementary to the single-stranded target sequence 1a, the first sequence 6a, and the second sequence 6b, respectively,

both of the forward primer 4 and the reverse primer 5 serve as an origin for elongation reaction with the DNA polymerase,

the forward primer 4 is complementary to a sequence 6c located at the 3′-end side of the single-stranded target sequence 1a,

the reverse primer 5 is complementary to a sequence 7c located at the 3′-end side of the complementary single-stranded target sequence 1b,

the first block nucleic acid 20 does not serve as an origin for elongation reaction with the DNA polymerase,

the first block nucleic acid 20 is complementary to a part of the third sequence 7a.

### ADVANTAGEOUS EFFECT OF INVENTION

The generation of the undesirable amplified double-stranded DNA sequence is suppressed.

### BRIEF DESCRIPTION OF THE FIGS

FIG. 1 shows a conventional PCR method.

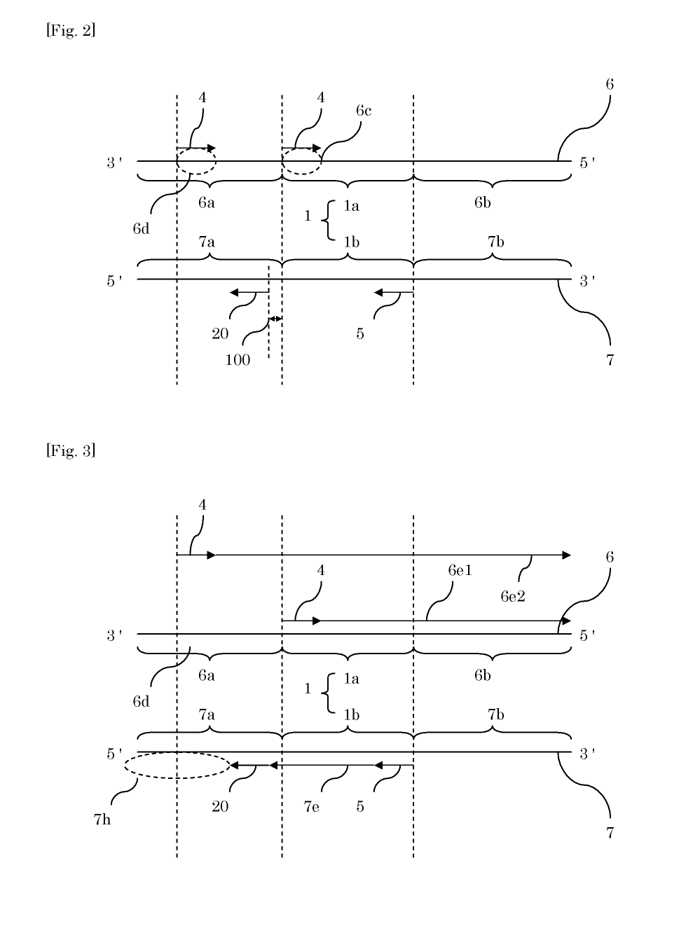

FIG. 2 shows the PCR method according to the present embodiment.

FIG. 3 shows the PCR method subsequent to FIG. 2 according to the present embodiment.

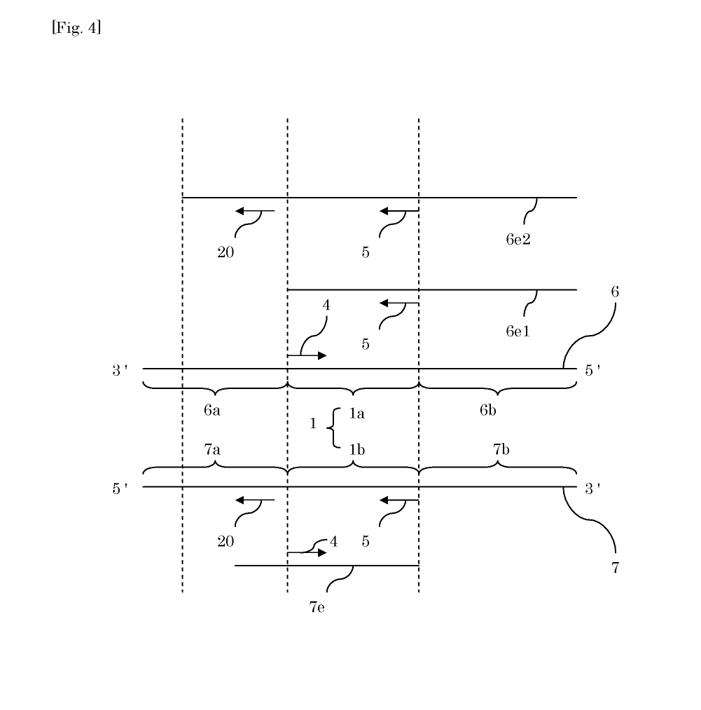

FIG. 4 shows the PCR method subsequent to FIG. 3 according to the present embodiment.

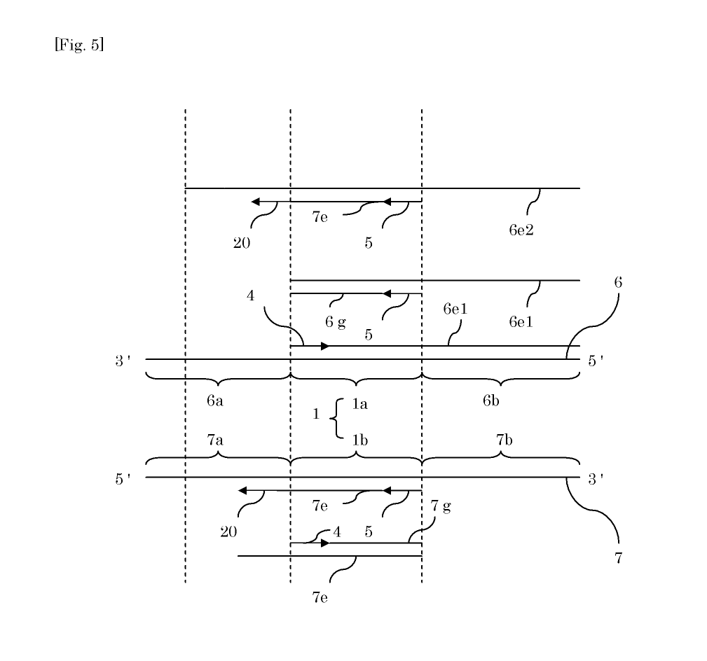

FIG. 5 shows the PCR method subsequent to FIG. 4 according to the present embodiment.

FIG. 6 shows the PCR method subsequent to FIG. 5 according to the present embodiment.

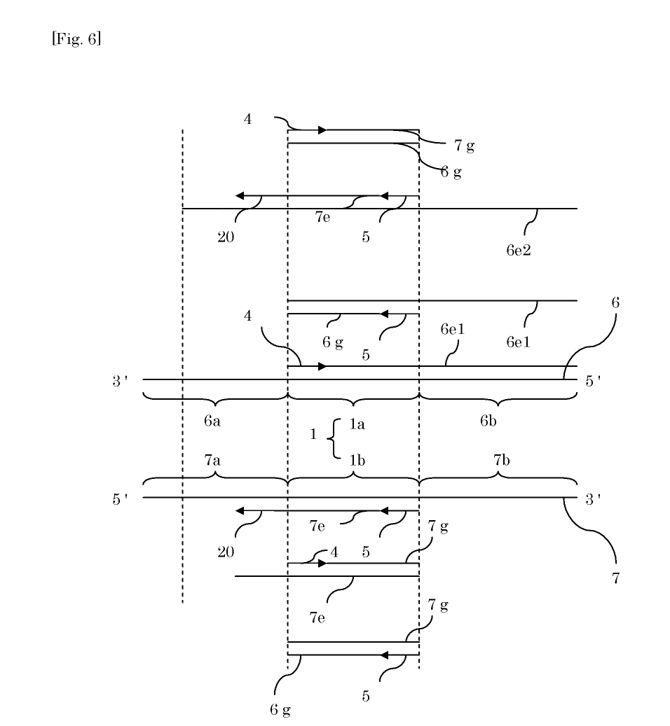

FIG. 7 shows a conventional PCR method.

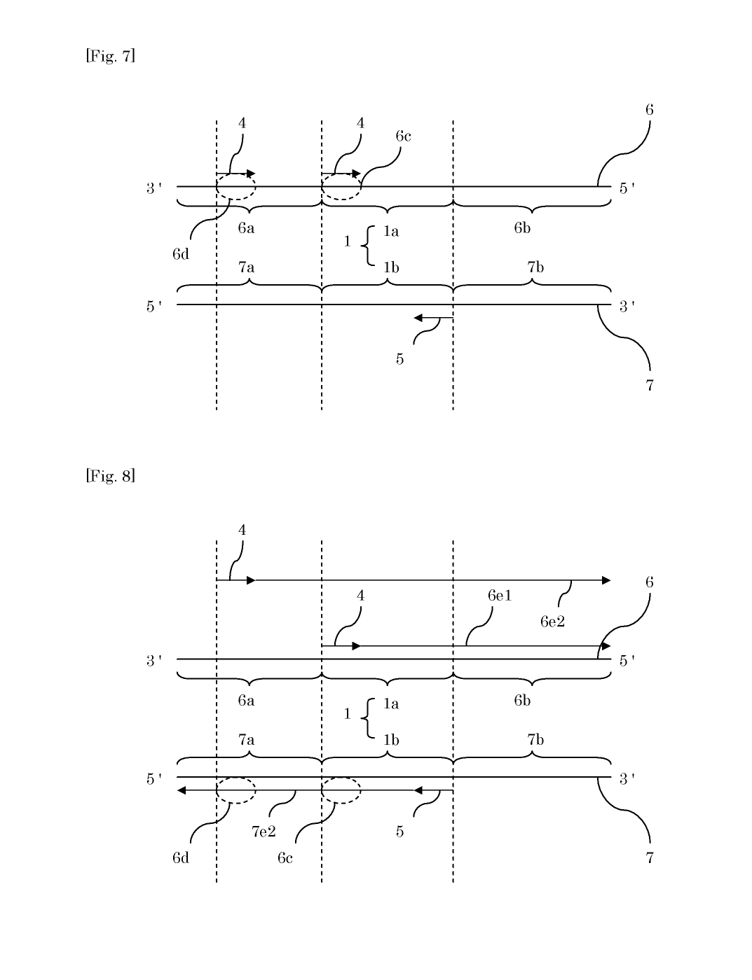

FIG. 8 shows the conventional PCR method subsequent to FIG. 7.

FIG. 9 shows the conventional PCR method subsequent to FIG. 8.

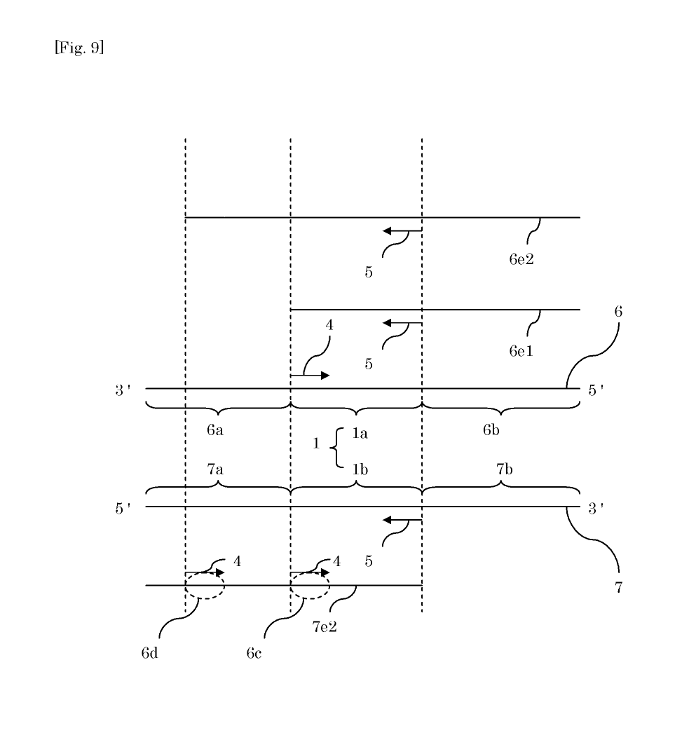

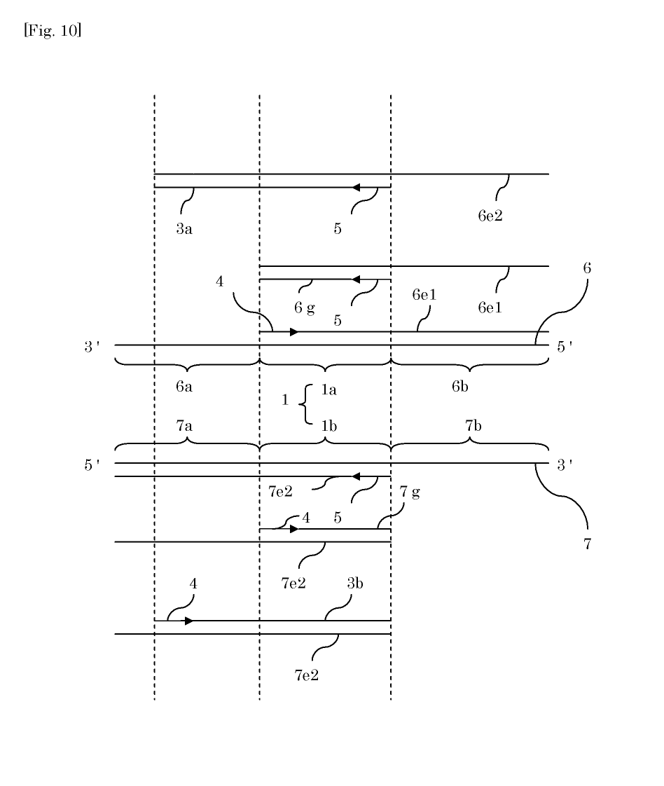

FIG. 10 shows the conventional PCR method subsequent to FIG. 9.

FIG. 11 shows the conventional PCR method subsequent to FIG. 10.

FIG. 12 shows the graph of the result of the electrophoresis in the example 1.

FIG. 13 shows the graph of the result of the electrophoresis in the comparative example 1.

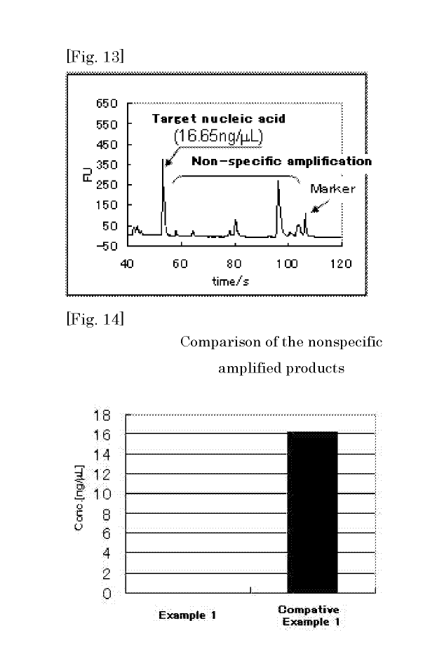

FIG. 14 shows a graph to compare the concentration of the nonspecifically amplified products in the example 1 and in the comparative example 1.

FIG. 15 shows the graph of the result of the electrophoresis in the example 2.

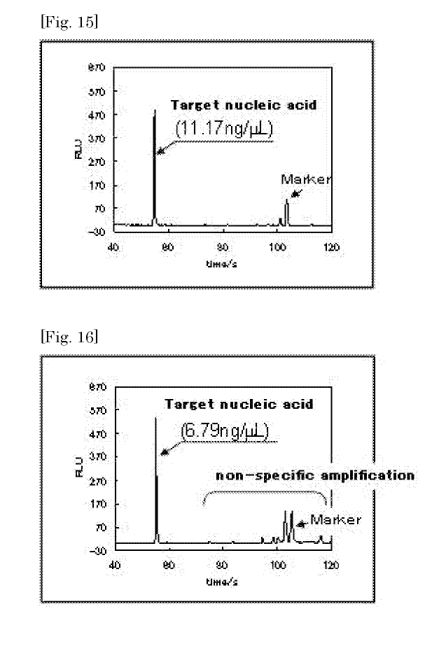

FIG. 16 shows the graph of the result of the electrophoresis in the comparative example 2.

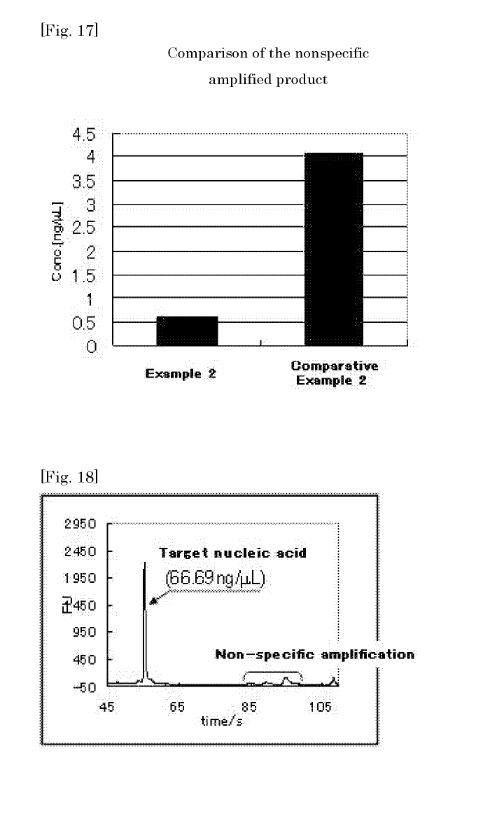

FIG. 17 shows a graph to compare the concentration of the nonspecifically amplified products in the example 2 and in the comparative example 2.

FIG. 18 shows the graph of the result of the electrophoresis in the example 3.

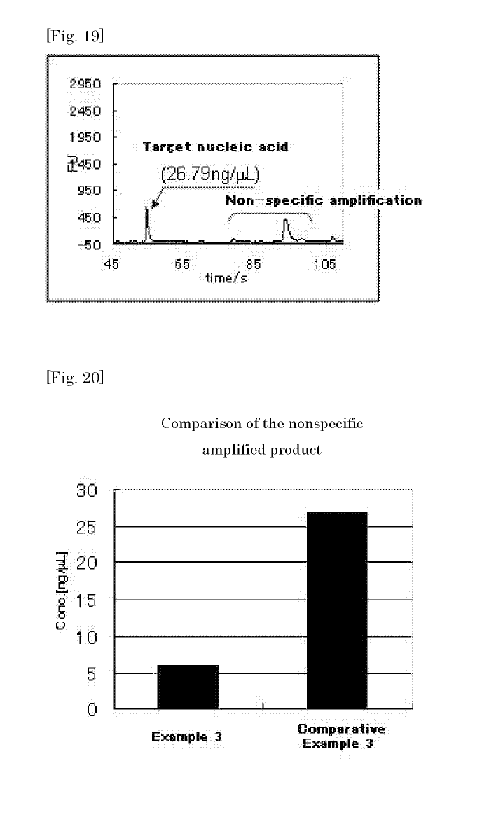

FIG. 19 shows the graph of the result of the electrophoresis in the comparative example 3.

FIG. 20 shows a graph to compare the concentration of the nonspecifically amplified products in the example 3 and in the comparative example 3.

### DESCRIPTION OF EMBODIMENT

The method according to the embodiment of the present invention is described below with reference to FIG. 2-FIG. 11.

### Embodiment

The present invention is characterized by adding a first block nucleic acid 20 on the amplification of the double-stranded target sequence 1 by a PCR method.

The first block nucleic acid 20 does not serve as an origin for elongation reaction with DNA polymerase. Preferably, the block nucleic acid is a synthetic oligonucleic acid.

An example of the first block nucleic acid 20 is a modified DNA, a modified Locked Nucleic Acid (hereinafter, referred to as “LNA”), or a peptide nucleic acid (hereinafter, referred to as “PNA”).

A nucleic acid is a biopolymer where a plurality of nucleotides were connected through phosphoester bonds. Each of the nucleotides is composed of a sugar molecule, a phosphate group, and a base. The OH group at the position 3 of the sugar molecule is substituted or modified with a hydrogen atom, a phosphate group, an amino group, a biotin group, a thiol group, or the derivatives thereof. The PNA does not require such a modification.

The forward primer 4 is complementary to the sequence 6c located at the 3′-end side of the single-stranded target sequence 1a. Accordingly, the forward primer 4 binds to the sequence 6c, as shown in FIG. 2. Furthermore, the forward primer 4 may bind to the sequence 6d which is included in the first sequence 6a and which is identical or similar to the sequence 6c.

The reverse primer 5 is complementary to the sequence 7c located at the 3′ end side of the complementary single-stranded target sequence 1b. Accordingly, the reverse primer 5 binds to the sequence 7c, as shown in FIG. 2.

The block nucleic acid 20 is complementary to the sequence 7y included in the third sequence 7a. Accordingly, the block nucleic acid 20 binds to the sequence 7y, as shown in FIG. 2.

When the PCR method starts, as shown in FIG. 3, DNAs are extended from the 3′-end of the forward primer 4 and from the 3′-end of the reverse primer 5 so as to form a first replication sequence 6e1, a second replication sequence 7e, and a third replication sequence 6e2.

The first replication sequence 6e1 is complementary to the sequence formed by continuously connecting the single-stranded target sequence 1a to the second sequence 6b. The third replication sequence 6e2 is complementary to the sequence formed by continuously connecting, the second sequence 6b, the single-stranded target sequence 1a, and a part of the first sequence 6a.

The block nucleic acid 20 stops the DNA elongation from the reverse primer 5. Accordingly, the second replication sequence 7e is complementary to the sequence formed by continuously connecting the complementary single-stranded target sequence 1b to a part of the third sequence 7a. However, the second replication sequence 7e does not include a sequence 7h complementary to the sequence interposed between the 5′-end of the sequence 7y and the 5′-end of the second single-stranded DNA 7.

When the PCR method is proceeded more, as shown in FIG. 4, the forward primer 4 binds to the first single-stranded DNA sequence 6 and the second replication sequence 7e. The reverse primer 5 binds to the second single-stranded DNA sequence 7, the first replication sequence 6e1, and the third replication sequence 6e2. The block nucleic acid 20 binds to the third sequence 7a and the third replication sequence 6e2.

Subsequently, as shown in FIG. 5, DNAs are extended from the 3′-ends of the two forward primers 4 to form the first replication sequence 6e1 and an amplified complementary single-stranded target sequences 7g. DNAs are extended from the 3′ ends of three reverse primers 5 to form one amplified single-stranded target sequence 6g and the two second replication sequence 7e. The amplified single-stranded target sequence 6g and the amplified complementary single-stranded target sequence 7g are identical to the single-stranded target sequence 1a and the complementary single-stranded target sequence 1b, respectively.

In other words, the first replication sequence 6e1 is formed from the first single-stranded DNA sequence 6 and the forward primer 4. The amplified single-stranded target sequences 6g is formed from the first replication sequence 6e1 and the reverse primer 5. The second replication sequence 7e is formed from the second replication sequence 6e2 and the reverse primer 5. Similarly, the second replication sequence 7e is formed from the second single-stranded DNA sequence 7 and the reverse primer 5. The amplified complementary single-stranded target sequence 7g is formed from the second replication sequence 7e and the forward primer 4.

When the PCR method is proceeded more, as shown in FIG. 6, not only the sequences formed in FIG. 5 but also the amplified complementary single-stranded target sequences 7g is formed from the amplified single-stranded target sequences 6g and the forward primer 4 in one cycle. Similarly, the amplified single-stranded target sequence 6g is formed from the amplified complementary single-stranded target sequence 7g and the reverse primer 5.

After cycles was repeated n times, the number of the amplified single-stranded target sequences 6g becomes 2ⁿ. Similarly, the number of the amplified complementary single-stranded target sequences 7g becomes 2ⁿ. On the contrary, an undesirable amplified double-stranded DNA sequence 3 does not exist. Of course, this is because of the block nucleic acid 20.

In FIG. 2, similarly to an ordinal PCR method, temperature is raised to unbind a double-stranded DNA. Subsequently, temperature is lowered to bind the forward primer 4 and the reverse primer 5 to the DNA sequence.

FIG. 7 shows a conventional PCR method, in which the block nucleic acid 20 is not used.

The forward primer 4 is complementary to the sequence 6c located at the 3′-end side of the single-stranded target sequence 1a. Accordingly, as shown in FIG. 7, the forward primer 4 binds to the sequence 6c. Furthermore, the forward primer 4 may bind not only to the sequence 6c but also to the sequence 6d, when the first sequence 6a includes the sequence 6d, which is identical or similar to the sequence 6c.

Unlike the PCR method shown in FIG. 2-FIG. 6, as shown in FIG. 8, after the conventional PCR method starts, formed is a second replication sequence 7e2 which is complementary to the sequence formed by connecting continuously the complementary single-stranded sequence 1b to the third sequence 7a. The second replication sequence 7e2 includes not only the sequence 6c but also the sequence 6d.

When the PCR method is proceeded more, as shown in FIG. 9, the forward primer 4 binds not only to the sequence 6c but also to the sequence 6d.

Accordingly, as shown in FIG. 10, not only the amplified single-stranded target sequence 6g and the amplified complementary single-stranded target sequences 7g but also the undesirable amplified single-stranded sequence 3a and the undesired amplified complementary single-stranded sequence 3b are formed. After the cycles are repeated n times, not only the 2ⁿ amplified single-stranded target sequences 6g and the 2ⁿ amplified complementary single-stranded target sequences 7g but also the 2ⁿ undesirable amplified single-stranded sequences 3a and the 2ⁿ undesired amplified complementary single-stranded sequences 3b are formed.

As is clear from FIG. 2-FIG. 6 and FIG. 7-FIG. 10, the block nucleic acid 20 inhibits the formation of the undesired amplified single-stranded sequence 3a and the amplified complementary single-stranded DNA sequence 3b, both of which consist of the undesired amplified double-stranded sequence 3. In this way, the generation of the undesirable amplified double-stranded DNA sequence 3 is suppressed.

As shown in FIG. 2, it is preferred that a sequence 100 interposed between the 5′-end of the block nucleic acid 20 and the 5′-end of the complementary target sequence 1b is a sequence consisting of bases of not less than 0 and not more than 20. This is because it is significantly undesirable that the sequence 100 includes the sequence 6d identical or similar to the sequence 6c. In other words, when the sequence 100 includes the sequence 6d, the forward primer 4 may binds to the sequence 6d by mistake to form the undesirable amplified double-stranded DNA sequence 3, as shown in FIG. 1.

As shown in FIG. 11, a second block nucleic acid 30 can be used. The second block nucleic acid 30 is complementary to a part of the second sequence 6b. Similarly to the first block nucleic acid 20, a block nucleic acid 30 consists of the modified DNA, the modified LNA, or the PNA.

As shown in FIG. 11, the second block nucleic acid 30 suppresses the generation of the undesirable amplified double-stranded DNA sequence 3 more efficiently together with the first block nucleic acid 20.

An example of the DNA polymerase used in the present invention is Taq DNA Polymerase or Pfu DNA Polymerase. It is preferable that the DNA polymerase does not have a 5′->3′exonuclease activity.

Deoxynucleoside triphosphate is a mixture of deoxyadenosine triphosphate (dATP), deoxythymidine triphosphate (dTTP), deoxyguanosine triphosphate (dGTP), and the deoxycytidine triphosphate (dCTP). These four kinds of compounds are included in an ordinal deoxynucleoside triphosphate equivalently. The normal deoxynucleoside triphosphate has a concentration of 20 μM-200 μM.

### EXAMPLES

Experiments of the present invention are described below.

### Example 1

In the example 1, the region of Exon6 of the human ABO blood group gene was amplified.

Table 1 shows the sequences of the forward primer 4 (hereinafter, referred to as “ABO-F”) and the reverse primer 5 (hereinafter, referred to as“ABO-R”) used in example 1.

TABLE 1

| | |
|---|---|
| ABO-F | 5′-TAGGAAGGATGTCCGCG-3′ |
| | (SEQ ID: 01) |
| ABO-R | 5′ TTCTTGATGGCAAACACAGT TAACC-3′ |
| | (SEQ ID: 02) |

Table 2 shows the sequence of the block nucleic acid 20 (hereinafter, referred to as “ABO-Block”).

TABLE 2

| | |
|---|---|
| ABO-Block | 5′-GTGCGGCCACATGGAGCTGGC-3′ |
| | (SEQ ID: 03) |

The target sequence of the 135 base pairs included in the ABO blood group genes of the type AB subject was amplified with the pair of the primers ABO-F and ABO-R.

The ABO-Block consists of a sequence complementary to the 201^{st}-221^{st} bases from 3′-end of the second single-stranded DNA 7. This sequence is also the 19^{th}-39^{th} bases from the 3′-end of the third sequence 7a included in the second single-stranded DNA 7. The carbon atom at the position 3 of the sugar molecule of the nucleotide at the 3′-end of the ABO-Block is modified with a phosphate group.

Using a DNA Micro Kit (available from QIAGEN company), genomic DNAs were extracted from 100 μL of the blood of the type AB subject so as to prepare a template DNA having a concentration of 10 ng/μL.

The reaction solution of the PCR contained the following chemical reagents.

1× TITANIUM Taq PCR buffer (available from Clontech company)

200 μM dNTP (dATP, dTTP, dGTP and dCTP mixture)

1× TITANIUM Taq DNA Polymerase (available from Clontech company)

1 μM ABO-F

1 μM ABO-R

0.5 ng/a of the genomic DNA, and

10 μM ABO-Block

Total volume: 10 μL

The PCR method was performed in accordance with the thermal profile shown in Table 3.

TABLE 3

| | |
|---|---|
| 1 cycle | At 95 degrees Celsius for one minute |

Cycle number: 1
Afterwards,

| | |
|---|---|
| 1 cycle | At 95 degrees Celsius for one second (for denaturation) |
| | At 62 degrees Celsius for one second (for annealing) |
| | At 72 degrees Celsius for one second (for DNA elongation) |

Cycle number: 50

After the PCR method was performed, the PCR reactant was subjected to electrophoresis using a BioAnalyzer (available from Agilent company). FIG. 12 shows the electrophoresis result. As is clear from FIG. 12, only the target sequence consisting of 135 base pairs was specifically amplified, and non-specific amplification did not occur. The marker indicated in FIG. 12 is a DNA Marker attached to the electrophoresis kit.

### Comparative Example 1

Except that the reaction solution containing the block nucleic acid in the example 1 was replaced with distilled water, the PCR method similar to that of the example 1 was carried out. FIG. 13 shows the electrophoresis result in the comparative example 1.

As is clear from FIG. 13, the target sequence consisting of 135 base pairs was amplified. However, other double-stranded DNA sequences were amplified nonspecifically. The other double-stranded DNA sequences consisted of 183-1209 base pairs.

As is clear from FIG. 12 and FIG. 13, the block nucleic acid 20 suppresses the non-specific amplification.

FIG. 14 is a graph showing the summation of the concentration of the non-specific amplified products calculated from the electrophoresis analysis result in the example 1 and the comparative example 1. As is clear from FIG. 14, the non-specific amplified products having a concentration of 16.15 ng/a were generated in the comparative example 1. On the contrary, in the example 1, the non-specific amplification was completely suppressed. It is believed that this is synergistic effect provided by suppressing the non-specific amplification. In other words, the number of the primers binding to the sequence 6d is reduced, and the number of the primers binding to the sequence 6c is increased. Accordingly, the target sequence is amplified efficiently. In this way, the block nucleic acid 20 contributes to the efficient amplification of the target sequence.

### Example 2

In the example 2, the region of the twelfth Exon of the human acetaldehyde dehydrogenase 2 gene was amplified.

Table 4 shows the sequences of the forward primer 4 (hereinafter, referred to as “ALDH2-F”) and the reverse primer 5 (hereinafter, referred to as “ALDH2-R”) used in the example 2.

TABLE 4

| | |
|---|---|
| ALDH2-F | 5′-GGCTGCAGGCATACACT-3′ |
| | (SEQ ID: 04) |
| ALDH2-R | 5′-GGCAGGTCCTGAACCTC -3′ |
| | (SEQ ID: 05) |

Table 5 shows the sequence of the block nucleic acid 20 (hereinafter, referred to as “ALDH2-Block”) used in the example 2.

TABLE 5

| | |
|---|---|
| ALDH2-Block | 5′-AGCAGTTGACCCTGTAATTTG-3′ |
| | (SEQ ID: 06) |

The target sequence consisting of 155 base pairs included in the human acetaldehyde dehydrogenase 2 gene was amplified with the pair of the primers consisting of the ALDH2-F and the ALDH2-R.

The ALDH2-Block consists of a sequence complementary to the 77th to 97th bases from the 3′ end of the third sequence 7a. The ALDH2-Block contains 21 bases. The carbon atom at the position 3 of the sugar of the nucleotide at the 3′-end of the ALDH2-Block is modified with a phosphate group.

Using a DNA Micro Kit (available from QIAGEN company), genomic DNAs were extracted from 100 μL of a human blood so as to prepare a template DNA having a concentration of 10 ng/μL.

The PCR reaction solution contained the following chemical reagents.

1×LA PCR buffer (available from TaKaRa company)

1.5 mM MgCl₂

200 μM dNTP (mixture of dATP, dTTP, dGTP, and dCTP)

0.05 U/10 μL LA Taq (available from TaKaRa company)

1 μM ALDH2-F

1 μM ALDH2-R

0.5 ng/μL of the genomic DNA, and

10 μM ALDH2-Block

Total volume: 10 μL

The PCR method was performed in accordance with the thermal profile shown in Table 6.

TABLE 6

| | |
|---|---|
| 1 cycle | At 95 degrees Celsius for one minute |

Cycle number: 1
Afterwards,

| | |
|---|---|
| 1 cycle | At 95 degrees Celsius for one second (for denaturation) |
| | At 58 degrees Celsius for one second (for annealing) |
| | At 72 degrees Celsius for one second (for DNA elongation) |

Cycle number: 30

After the PCR method was carried out, the PCR reactant was subjected to electrophoresis similarly to the example 1. FIG. 15 shows the electrophoresis result. As is clear from FIG. 15, the target sequence consisting of 155 base pairs was amplified efficiently.

### Comparative Example 2

Except that the reaction solution containing the block nucleic acid in the example 2 was replaced with distilled water, the PCR method similar to that of the example 2 was carried out. FIG. 16 shows the electrophoresis result in the comparative example 2.

As is clear from FIG. 16, the target sequence consisting of 155 base pairs was amplified. However, other double-stranded DNA sequences were amplified nonspecifically. The other double-stranded DNA sequences consisted of 665-1252 base pairs.

FIG. 17 is a graph showing the summation of the concentration of the non-specific amplified products calculated from the electrophoresis analysis result in the example 2 and the comparative example 2. As is clear from FIG. 17, the non-specific amplified products having a concentration of 4.07 ng/a was generated in the comparative example 2. On the contrary, in the example 2, the concentration is decreased to 0.6 ng/μL. Similarly to the example 1, this means that the block nucleic acid 20 contributes to the efficient amplification of the target sequence.

### Example 3

In the example 3, the human dystrophin gene was amplified.

Table 7 shows the sequences of the forward primer 4 (hereinafter, referred to as “Dys-F”) and the reverse primer 5 (hereinafter, referred to as “Dys-R”) used in example 3.

TABLE 7

| | |
|---|---|
| Dys-F | 5′-AGGCTTGAAAGGGCAAGTAGAAGT-3′ |
| | (SEQ ID: 07) |
| Dys-R | 5′-GCTGATCTGCTGGCATCTTGC-3′ |
| | (SEQ ID: 08) |

Table 8 shows the sequences of the first block nucleic acid 20 (hereinafter, referred to as “Dys-Block-1”) and the second block nucleic acid 30 (hereinafter, referred to as “Dys-Block-2”) used in the example 3.

TABLE 8

| | |
|---|---|
| Dys-Block-1 | 5′-GACTTTTTCTCAACACTTTTGCCATC-3′ |
| | (SEQ ID: 09) |
| Dys-Block-2 | 5′-TGGAAGAAAATGGGATGTGGTAGAA-3′ |
| | (SEQ ID: 10) |

The target sequence consisting of 147 base pairs included in the human dystrophin gene was amplified with the pair of the primers consisting of the Dys-F and the Dys-R.

The Dys-Block-1 consists of a sequence complementary to the 40th to 65th bases from the 3′ end of the third sequence 7a. The Dys-Block-1 contains 26 bases. Dys-Block-2 consists of a sequence complementary to the 222^{nd} to 246^{th} bases from the 3′ end side of the second sequence 6b. The Dys-Block-2 contains 25 bases.

The carbon atom at the position 3 of the sugar molecule included in the nucleotide of the 3′-end of the Dys-Block-1 was modified with a phosphate group. Similarly, the carbon atom at the position 3 of the sugar molecule of the nucleotide at the 3′-end of the Dys-Block-2 was modified with a phosphate group.

Using a DNA Micro Kit (available from QIAGEN company), genomic DNAs were extracted from 100 μL of human blood so as to prepare a template DNA having a concentration of 10 ng/μL.

The PCR reaction solution contained the following chemical reagents.

1× TITANIUM Taq PCR buffer (available from Clontech company)

200 μM dNTP (mixture of dATP, dTTP, dGTP, and dCTP)

1× TITANIUM Taq DNA Polymerase (available from Clontech company)

1 μM Dys-F

1 μM Dys-R

0.5 ng/μL of the genomic DNA

10 μM Dys-Block-1, and

10 μM Dys-Block-2

Total volume: 10 μL

The PCR method was performed in accordance with the thermal profile shown in Table 9.

TABLE 9

| | |
|---|---|
| 1 cycle | At 95 degrees Celsius for one minute |

Cycle number: 1
Afterwards,

| | |
|---|---|
| 1 cycle | At 95 degrees Celsius for one second (for denaturation) |
| | At 54 degrees Celsius for one second (for annealing) |
| | At 72 degrees Celsius for one second (for DNA elongation) |

Cycle number: 50

After the PCR method was performed, the PCR reactant was subjected to electrophoresis in the same way as that of the example 1. FIG. 18 shows electrophoresis result. As is clear from FIG. 18, the target sequence consisting of 147 base pairs was amplified efficiently.

### Comparative Example 3

Except that the reaction solution containing the block nucleic acid in the example 3 was replaced with distilled water, the PCR method in the same way as that of the example 3 was carried out. FIG. 19 shows the electrophoresis result in the comparative example 3.

As is clear from FIG. 19, the target sequence consisting of 147 base pairs was amplified. However, other double-stranded DNA sequences were amplified nonspecifically. The other double-stranded DNA sequences consisted of 375-712 base pairs.

FIG. 20 is a graph showing the summation of the concentration of the non-specific amplified products calculated from the electrophoresis analysis result in the example 3 and the comparative example 3. As is clear from FIG. 20, the non-specific amplified products having a concentration of 26.93 ng/μL was generated in the comparative example 3. On the contrary, in the example 3, the concentration is decreased to 5.97 ng/μL. Similarly to the example 1 and the example 2, this means that the first block nucleic acid 20 and the second block nucleic acid 30 contribute to the efficient amplification of the target sequence.

### INDUSTRIAL APPLICABILITY

The present invention can be used in a general PCR method. The present invention can be used in a PCR method for laboratory studies.

### REFERENCE SIGNS LIST

1: double-stranded target sequence
   1a: single-stranded target sequence
   1b: complementary single-stranded target sequence
2: desirable amplified double-stranded DNA
3: undesirable amplified double-stranded DNA
   3a: undesirable amplified single-stranded DNA
   3a: undesirable amplified complementary single-stranded DNA
4: forward primer
5: reverse primer
6: first single-stranded DNA
   6a: first sequence
   6b: second sequence
   6c: sequence located at the 3′-end side of the single-stranded target sequence 1a
   6d: sequence identical or similar to the sequence 6c
   6e1: first replication sequence
   6e2: third replication sequence
   6g: amplified single-stranded target sequence
7: second single-stranded DNA
   7a: third sequence
   7b: fourth sequence
   7c: sequence located at the 3′-end side of the complementary single-stranded target sequence 1b
   7e: second replication sequence
   7e2: second replication sequence
   7g: amplified complementary single-stranded target sequence
   7y: sequence included in the third sequence 7a
20: first block nucleic acid
30: second block nucleic acid

### SEQUENCE LISTING FREE TEXT

SEQ ID: 1: Forward primer for amplifying the human ABO blood group gene

SEQ ID: 2: Reverse primer for amplifying the human ABO blood group gene

SEQ ID: 3: Oligonucleic acid (DNA) for suppressing the nonspecific amplification

SEQ ID: 4: Forward primer for amplifying the human acetaldehyde dehydrogenase 2 gene

SEQ ID: 5: Reverse primer for amplifying the human acetaldehyde dehydrogenase 2 gene

SEQ ID: 6: Oligonucleic acid (DNA) for suppressing the nonspecific amplification

SEQ ID: 7: Forward primer for amplifying the human dystrophin gene

SEQ ID: 8: Reverse primer for amplifying the human dystrophin gene

SEQ ID: 9: Oligonucleic acid (DNA) for suppressing the nonspecific amplification

SEQ ID: 10: Oligonucleic acid (DNA) for suppressing the nonspecific amplification

1. A method for amplifying a double-stranded target sequence included in a double-stranded DNA, the method comprising steps of:
a step (a) of mixing DNA polymerase, deoxynucleoside triphosphate, the double-stranded DNA, a forward primer, a reverse primer, and a first block nucleic acid 20, and amplifying the double-stranded target sequence using a PCR method, wherein,
the double-stranded DNA consists of a first single-stranded DNA 6 and a second single-stranded DNA 7,
the double-stranded target sequence consist of a single-stranded target sequence 1a and a complementary single-stranded target sequence 1b,
the first single-stranded DNA 6 consists of 3′ end—a first sequence 6a—the single-stranded target sequence 1a—a second sequence 6b—5′ end, the second single-stranded DNA 7 consists of 5′-end—a third sequence 7a—the complementary single-stranded target sequence 1b—a fourth sequence 7b—3′-end,
the complementary single-stranded target sequence 1b, the third sequence 7a, and the fourth sequence 7b are complementary to the single-stranded target sequence 1a, the first sequence 6a, and the second sequence 6b, respectively,
both of the forward primer 4 and the reverse primer 5 serve as an origin for elongation reaction with the DNA polymerase,
the forward primer 4 is complementary to a sequence 6c located at the 3′-end side of the single-stranded target sequence 1a,
the reverse primer 5 is complementary to a sequence 7c located at the 3′-end side of the complementary single-stranded target sequence 1b,
the first block nucleic acid 20 does not serve as an origin for elongation reaction with the DNA polymerase,
the first block nucleic acid 20 is complementary to a part of the third sequence 7a.

2. The method according to claim 1, wherein
the first block nucleic acid consists of a DNA where an OH group at a position 3 of a sugar molecule included in a nucleotide located at the 3′-end thereof is substituted or modified with a hydrogen atom, a phosphate group, an amino group, a biotin group, a thiol group, or the derivative thereof.

3. The method according to claim 1, wherein
the first block nucleic acid consists of a Locked Nucleic Acid where an OH group at a position 3 of a sugar molecule included in a nucleotide located at the 3′-end thereof is substituted or modified with a hydrogen atom, a phosphate group, an amino group, a biotin group, a thiol group, or the derivative thereof.

4. The method according to claim 1, wherein
The first block nucleic acid consists of a peptide nucleic acid.

5. The method according to claim 1, wherein
in the step (a), a second block nucleic acid is added,
the second block nucleic acid does not serves as an origin for elongation reaction with the DNA polymerase, and
the second block nucleic acid is complementary with a part of the second sequence.

6. The method according to claim 5, wherein
The second block nucleic acid consists of a DNA where an OH group at a position 3 of a sugar molecule included in a nucleotide located at the 3′-end thereof is substituted or modified with a hydrogen atom, a phosphate group, an amino group, a biotin group, a thiol group, or the derivative thereof.

7. The method according to claim 5, wherein
The second block nucleic acid consists of a Locked Nucleic Acid where an OH group at a position 3 of a sugar molecule included in a nucleotide located at the 3′-end thereof is substituted or modified with a hydrogen atom, a phosphate group, an amino group, a biotin group, a thiol group, or the derivative thereof.

8. The method according to claim 5, wherein
The second block nucleic acid consists of a peptide nucleic acid.
